(19) United States
(12) Patent Application Publication
(10) Pub. No.: US 2017/0370011 A1
(43) Pub. Date: Dec. 28, 2017

(54) METHOD OF PREPARING OXALIC ACID

(71) Applicant: SHELL OIL COMPANY, HOUSTON, TX (US)

(72) Inventor: Jan Hermen Hendrik MEURS, Amsterdam (NL)

(21) Appl. No.: 15/548,110

(22) PCT Filed: Feb. 3, 2016
(86) PCT No.: PCT/EP2016/052289

§ 371 Date: Aug. 2, 2017

(30) Foreign Application Priority Data
Feb. 4, 2015 (EP) 15153770.1

(51) Int. Cl. C25B 3/04; C01D 7/10; B01D 61/42; C07C 29/149

(52) U.S. Cl. CPC C25B 3/04; C07C 29/149; C01D 7/10; B01D 61/422; B01D 2311/02; B01D 2311/263

(57) ABSTRACT

The present invention provides a method of preparing oxalic acid (H₂C₂O₄), the method at least comprising the steps of: (a) providing a metal formate (HCO₂M) containing stream, wherein the metal (M) of the metal formate (HCO₂M) is a monovalent metal selected from the group consisting of Li, Na, K, Cs, Rb and a mixture thereof; (b) heating the metal formate (HCO₂M) containing stream thereby obtaining a metal oxalate (M₂C₂O₄) containing stream; (c) subjecting the metal oxalate (M₂C₂O₄) containing stream to electrodialysis, thereby obtaining at least oxalic acid (M₂C₂O₄) and a metal hydroxide (MOH).

The present invention relates to a method of preparing oxalic acid (H₂C₂O₄). Various methods of preparing oxalic acid are known in the art.

One example of preparing oxalic acid is by reacting calcium oxalate with excess sulphuric acid (H₂SO₄). A problem of this known method is the high amount of sulphuric acid and calcium hydroxide needed, as well as the co-production of large amounts of gypsum (CaSO₄).

It is an object of the present invention to overcome or minimize the above problem.

It is a further object of the present invention to provide an alternative method of preparing oxalic acid.

It is an even further object to provide a new route to prepare ethylene glycol on the basis of oxalic acid, more in particular wherein the oxalic acid has been obtained using CO₂.

One or more of the above or other objects can be achieved by providing a method of preparing oxalic acid (H₂C₂O₄), the method at least comprising the steps of:

(a) providing a metal formate (HCO₂M) containing stream, wherein the metal (M) of the metal formate (HCO₂M) is a monovalent metal selected from the group consisting of Li, Na, K, Cs, Rb and a mixture thereof;
(b) heating the metal formate (HCO₂M) containing stream thereby obtaining a metal oxalate (M₂C₂O₄) containing stream;
(c) subjecting the metal oxalate (M₂C₂O₄) containing stream to electrodialysis, thereby obtaining at least oxalic acid (H₂C₂O₄) and a metal hydroxide (MOH).

It has surprisingly been found according to the present invention that oxalic acid can be prepared with a good yield in an effective manner, without requiring sulphuric acid or another strong acid and without the co-production of gypsum or other material that is to be disposed of.

A further advantage of the present invention is that no metal is consumed as it can be recycled to the metal formate salt.

It is of note that RO106878 discloses a method of preparing oxalic acid using an ion-exchange column (but is mute about electrodialysis).

In step (a), a metal formate (HCO₂M) containing stream is provided, wherein the metal (M) of the metal formate (HCO₂M) is a monovalent metal selected from the group consisting of Li (lithium), Na (sodium), K (potassium), Cs (cesium), Rb (rubidium) and a mixture thereof. Apart from the presence of a selected monovalent metal, this metal formate (HCO₂M) containing stream is not limited in any way. Preferably, the metal (M) of the metal formate (HCO₂M) is selected from the group consisting of K, Cs, Rb and a mixture thereof. More preferably, the metal formate (HCO₂M) is potassium formate (HCO₂K); hence, in the latter case the metal is potassium (K). By using a metal selected from the group consisting of K, Cs and Rb, and in particular K, a water-soluble formate (and oxalate) is obtained thereby minimizing the occurrence of precipitation. Preferably the water-soluble oxalate salt has a solubility of at least 20.0 g/l as determined at 20° C.

Typically, the metal formate (HCO₂M) containing stream comprises from 20 to 100 wt. % (based on dry matter) metal formate, preferably above 60 wt. %. Usually, the metal formate (HCO₂M) containing stream comprises some water and up to 10 wt. % (based on dry matter) metal hydroxide.

The person skilled in the art will readily understand that the metal formate (HCO₂M) can be obtained in various ways. As an example the metal formate (HCO₂M) can be obtained by reacting a metal hydroxide (MOH) with carbon monoxide (CO) or by the electrochemical reduction of the corresponding carbonate. According to a preferred embodiment according to the present invention, the metal formate (HCO₂M) has been obtained from a metal bicarbonate (MHCO₃), in particular by reacting the metal bicarbonate (MHCO₃) with hydrogen (H₂). As the person skilled in the art is familiar with the conversion of metal bicarbonate (MHCO₃) into metal formate (HCO₂M), this is not discussed here in detail. Typically, such a conversion is performed at a temperature in the range of from 60° C. to 200° C.

The person skilled in the art will readily understand that the metal bicarbonate (MHCO₃) can be obtained in various ways. According to an especially preferred embodiment of the present invention, the metal bicarbonate (MHCO₃) has been obtained from CO₂ and a metal hydroxide (MOH). As the person skilled in the art is familiar with the conversion of CO₂ and a metal hydroxide (MOH) into metal bicarbonate (MHCO₃), this is not discussed here in detail. As mere examples of such a conversion, reference is made in this respect to: (for conversion by hydrogenation) Y. Himeda, N. Onozowa-Komatsuzaki, H. Sugihara and K. Kasuga, J. Am. Chem. Soc., 2005, 127 (38), 13118; and (for conversion by electrochemical reduction) A. S. Agarwal, Y. Zhai, D. Hill and N. Sridhar, ChemSusChem 2011, 4, 1301-1310.

According to a preferred embodiment of the present invention, the metal hydroxide (MOH) obtained in step (c) is recycled for use in obtaining the metal bicarbonate (MHCO₃). Herewith the consumption of metal is minimized. In step (b), the metal formate (HCO₂M) containing stream is heated thereby obtaining a metal oxalate (M₂C₂O₄) containing stream. As the person skilled in the art is familiar with the conversion of metal formate (HCO₂M) into metal oxalate (M₂C₂O₄), this is not discussed here in detail; as a mere example, obtaining potassium oxalate from potassium formate using heating has been described in DE 660473. Typically, the metal formate (HCO₂M) containing stream is heated to a temperature of from 100 to 500° C., preferably above 250° C. and preferably below 480° C.

Typically, the metal oxalate (M₂C₂O₄) containing stream obtained in step (b) comprises at least 70 wt. % (based on dry matter) metal oxalate (M₂C₂O₄), preferably at least 80 wt. %, more preferably at least 90 wt. %. Usually, the metal oxalate (M₂C₂O₄) containing stream obtained in step (b) comprises small residual amounts of metal formate (preferably less than 5 wt. %, based on dry matter), metal carbonate (preferably less than 5 wt. %, based on dry matter) and metal hydroxide (preferably less than 10 wt. %, based on dry matter), the amount of water being variable.

In step (c), the metal oxalate (M₂C₂O₄) containing stream is subjected to electrodialysis, thereby obtaining at least oxalic acid (H₂C₂O₄) and a metal hydroxide (MOH).

As the person skilled in the art is familiar with electrodialysis, this is not discussed here in detail. As an example, the electrodialysis technique has been described in for example:
Fernando Valero, Angel Barceló and Ramon Arbós (2011). Electrodialysis Technology—Theory and Applications, Desalination, Trends and Technologies, Michael Schorr (Ed.), ISBN: 978-953-307-311-8;
Bernardes, Andrea, Siqueira Rodrigues, Marco Antonio, Zoppas Ferreira, Jane (Eds.), Electrodialysis and Water Reuse, Novel Approaches, Series: Topics in Mining, Metallurgy and Materials Engineering, ISBN 978-3-642-40249-4; and
Membrane Technology and Applications, Third Edition, Richard W. Baker, Published Online: 18 Jul. 2012, DOI: 10.1002/9781118359686.ch10, John Wiley & Sons, Ltd.

Typically, the temperature during the electrodialysis is from 0 to 150° C., preferably above 30° C. and preferably below 90° C.

Preferably, the metal oxalate (M₂C₂O₄) containing stream subjected in step (c) comprises at most 10.0 wt. % (based on dry matter) carbonate.

According to an especially preferred embodiment of the present invention, the oxalic acid (H₂C₂O₄) obtained in step (c) is reacted with hydrogen (H₂) to obtain ethylene glycol (HOCH₂CH₂OH).

As the person skilled in the art is familiar with the process of hydrogenation, this is not discussed here in detail. As an example, the a hydrogenation reaction has been described in J. E. Carnahan, T. A. Ford, W. F. Gresham, W. E. Grigsby and G. F. Hager, J. Am. Chem. Soc., 1953, 77, 3766.

In another aspect, the present invention provides a method of preparing ethylene glycol (HOCH₂CH₂OH) from CO₂, the method at least comprising the steps of:

(i) reacting CO₂ and a metal hydroxide (MOH) thereby obtaining a metal bicarbonate (MHCO₃), wherein the metal (M) of the metal hydroxide (MOH) is a monovalent metal selected from the group consisting of Li, Na, K, Cs, Rb and a mixture thereof;
(ii) reacting the metal bicarbonate (MHCO₃) obtained in step (i) with hydrogen (H₂) thereby obtaining a metal formate (HCO₂M);
(iii) heating the metal formate thereby obtaining a metal oxalate (M₂C₂O₄);
(iv) subjecting the metal oxalate (M₂C₂O₄) to electrodialysis, thereby obtaining at least oxalic acid (H₂C₂O₄) and a metal hydroxide (MOH);
(v) reacting the oxalic acid (H₂C₂O₄) obtained in step (iv) with hydrogen (H₂) to obtain ethylene glycol (HOCH₂CH₂OH).

An important advantage of this method of preparing ethylene glycol (HOCH₂CH₂OH) from CO₂, is that the co-production of by-products is minimized.

The steps (i) to (v) have been discussed above in relation to the method of preparing oxalic acid (H₂C₂O₄) and any preferences indicated earlier for the method of preparing oxalic acid (H₂C₂O₄) also apply for the method of preparing ethylene glycol (HOCH₂CH₂OH). Hence, again, it is preferred that the metal (M) is selected from the group consisting of K, Cs, Rb and a mixture thereof, and is preferably K. Also, it is preferred that at least part of the metal hydroxide (MOH) obtained during dialysis (in step (iv)) is reused, viz. in step (i).

Hereinafter the invention will be further illustrated by the following non-limiting examples.

### EXAMPLES

### Example 1

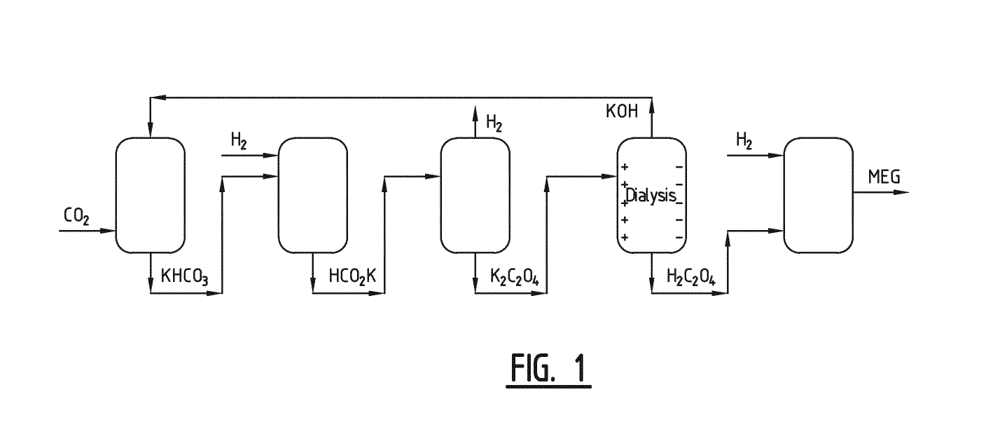

This Example 1 illustrates in general terms how ethylene glycol (HOCH₂CH₂OH) can be prepared from CO₂. A schematic overview of the preparation method of Example 1 is depicted in FIG. 1.

a. Preparation of Metal Bicarbonate (MHCO₃)

CO₂ and potassium hydroxide (KOH) are reacted, e.g. as described by R. V. Williamson and J. H. Mathews in Ind. Eng. Chem., 1924, 1157, thereby obtaining potassium bicarbonate (KHCOJ.

b. Preparation of Metal Formate (HCO₂M)

The potassium bicarbonate (KHCO₃) obtained is subsequently reacted with hydrogen (H₂), e.g. as described by Ryo Tanaka, Makoto Yamashita and Kyoko Nozaki in J. Am. Chem. Soc., 2009, 131, 14168, thereby obtaining potassium formate (HCO₂K).

c. Preparation of Metal Oxalate (M₂C₂O₄)

The potassium formate (HCO₂K) is heated, e.g. as described in DE 660473, thereby obtaining potassium oxalate (K₂C₂O₄).

d. Preparation of Oxalic Acid (H₂C₂O₄)

Subsequently, the potassium oxalate (K₂C₂O₄) is subjected to electrodialysis, thereby obtaining at least oxalic acid (H₂C₂O₄) and potassium hydroxide (KOH). The KOH can be recycled for use in the preparation of the potassium bicarbonate described above.

e. Preparation of Ethylene Glycol (HOCH₂CH₂OH; ‘MEG’)

The oxalic acid (H₂C₂O₄) may then be reacted with hydrogen (H₂) to obtain ethylene glycol (HOCH₂CH₂OH).

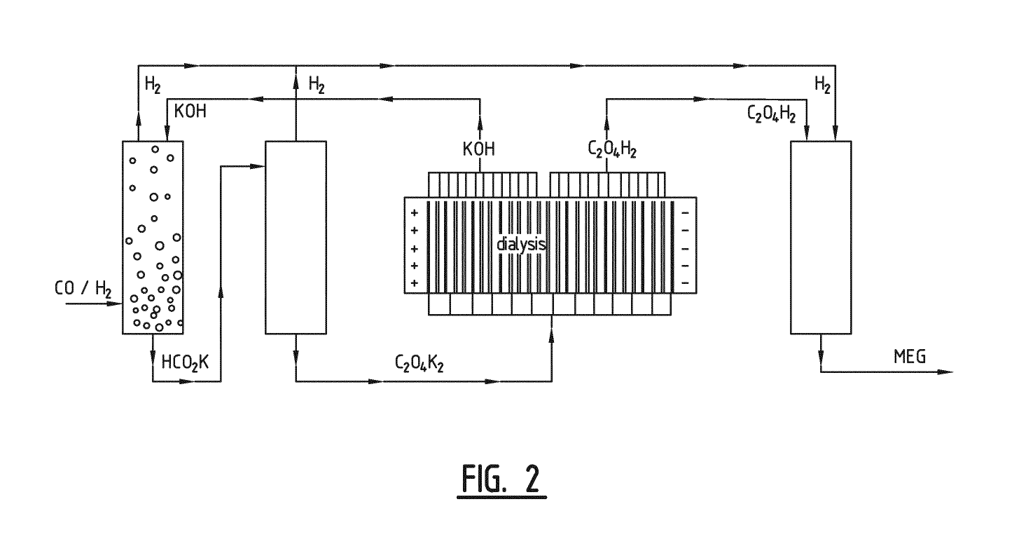

FIG. 2 schematically shows an alternative method according to the present invention for preparing ethylene glycol (HOCH₂CH₂OH), wherein the metal formate (i.c. HCO₂K) can be obtained by reacting a metal hydroxide (i.c. KOH) with carbon monoxide (CO).

### Example 2

Example 2 describes in further detail how oxalic acid (H₂C₂O₄) and potassium hydroxide (KOH) can be obtained by subjecting potassium oxalate (K₂C₂O₄) to electrodialysis.

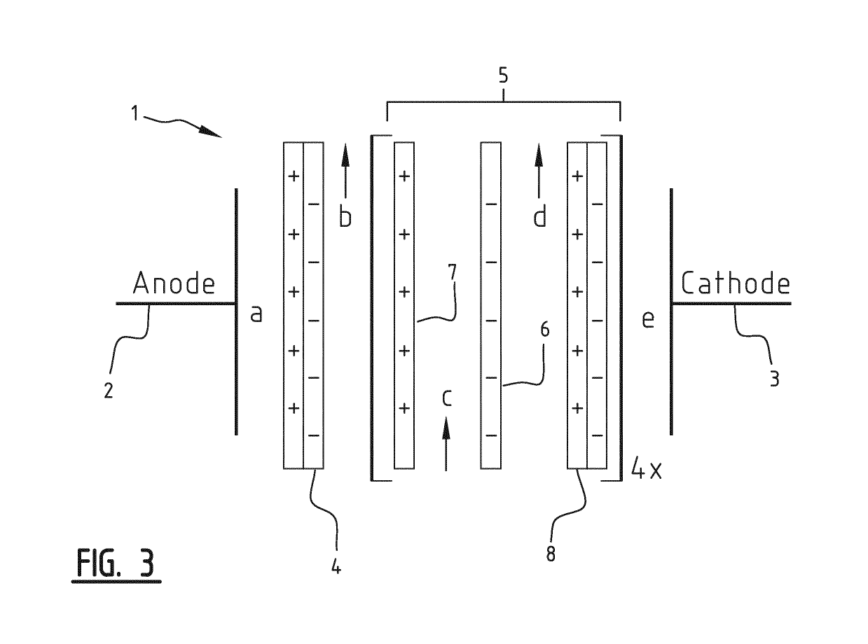

An electrodialysis cell as depicted in FIG. 3 was used. Electrodialysis cells are commercially available and can be obtained from e.g. Astom Corporation (Tokyo, Japan) or Fumatech GmbH (Bietigheim-Bissingen, Germany). The electrodialysis cell (referred to with 1 in FIG. 3) contained an anode 2 and a cathode 3. Between the anode 2 and cathode 3 a bipolar membrane 4 (Fumatech FBP, commercially available from Fumatech GmbH) and four repeating membrane stacks 5 were placed, thereby creating anode compartment a, cathode compartment e and further compartments b-d. Each membrane stack 5 consisted of an anion-exchange membrane 6 (Mega AMH, commercially available from Mega A.S. (Prague, Czech Republic)), a cation-exchange membrane 7 (Mega CMH, commercially available from Mega A.S.), and a bipolar membrane 8 (Fumatech FBP). The effective membrane surface was 100 cm².

Diluted sulphuric acid was used for the anode compartment a and a sodium hydroxide solution for the cathode compartment e; both the diluted sulphuric acid and the sodium hydroxide solution had a conductivity of 20 mS/cm.

A potassium oxalate containing stream (which was obtained by heating potassium formate according to DE 660473; containing 900 g potassium oxalate, 40 g potassium formate and 50 g potassium carbonate in 2.7 kg water) was fed into the compartments c (i.e. between the anion-exchange membrane 6 and the cation-exchange membrane 7 in the stacks 5) thereby generating an oxalic acid in compartment b and potassium hydroxide in compartment d. The fluids were circulated through the respective compartments at a flow of 20-50 l/h. The electrodialysis cell was operated at ambient temperature to 35° C. at a constant voltage of 20V.

The experiment was stopped when the current density dropped due to exhaustion of the feed; this started happening after about 6 hours, when the current dropped to below 5 mA/cm². Then the various streams were analysed.

Table 1 lists the amounts of potassium, oxalate, formate and carbonate (in g/kg (based on dry matter)) in the various streams. Further, Table 2 lists the current in mA/cm² at indicated times.

TABLE 1

| | Potassium | Oxalate | Formate | Carbonate |
|---|---|---|---|---|
| | [g/kg] | [g/kg] | [g/kg] | [g/kg] |
| Feed | 121.00 | 128.00 | 4.94 | 6.00 |
| (compartment C) | | | | |
| Feed, after dialysis | 1.69 | 1.56 | 0.03 | 0.53 |
| Oxalic acid stream | 0.41 | 66.40 | 3.19 | 0.04 |
| (compartment B) | | | | |
| Hydroxide stream | 69.00 | 0.67 | 0.06 | 0.44 |
| (compartment D) | | | | |

TABLE 2

| | Current [mA/cm²] | | |
|---|---|---|---|
| Time [hour] | Example 2 | Example 3 | Example 4 |
| 0 | 18 | 21 | 18 |
| 1 | 31 | 35 | 30 |
| 2 | 38 | 41 | 32 |
| 3 | 41 | 39 | 3 |
| 4 | 41 | 26 | — |
| 5 | 42 | 7 | — |
| 6 | 24 | — | — |
| 7 | 8 | — | — |

### Examples 3 and 4

Example 2 was repeated with two different feed streams (and resulting product streams) with the composition as given in Table 3 (Example 3) and Table 4 (Example 4) to show that the present invention works under various conditions. Table 2 above lists current in mA/cm² at indicated times.

TABLE 3

| | Potassium | Oxalate | Formate | Carbonate |
|---|---|---|---|---|
| | [g/kg] | [g/kg] | [g/kg] | [g/kg] |
| Feed | 60.5 | 64.00 | 2.47 | 3.00 |
| (compartment C) | | | | |
| Feed, after dialysis | 0.28 | 0.54 | 0.10 | 0.004 |
| Oxalic acid stream | 0.56 | 54.20 | 2.34 | 0.04 |
| (compartment B) | | | | |
| Hydroxide stream | 48.5 | 0.22 | 0.20 | 0.22 |
| (compartment D) | | | | |

TABLE 4

| | Potassium | Oxalate | Formate | Carbonate |
|---|---|---|---|---|
| | [g/kg] | [g/kg] | [g/kg] | [g/kg] |
| Feed | 30.30 | 32.00 | 1.25 | 1.50 |
| (compartment C) | | | | |
| Feed, after dialysis | 0.02 | 0.06 | 0.00 | 0.04 |
| Oxalic acid stream | 0.22 | 30.00 | 1.31 | 0.04 |
| (compartment B) | | | | |
| Hydroxide stream | 26.00 | 0.15 | 0.03 | 0.41 |
| (compartment D) | | | | |

### DISCUSSION

As can be seen from the Examples, the present invention provides a method for preparing oxalic acid, without the co-production of gypsum or other waste materials to be disposed of and without the need of large amounts of sulphuric acid. Further, the present invention provides a method of preparing ethylene glycol on the basis of oxalic acid and even a method of preparing ethylene glycol starting from CO₂.

The person skilled in the art will readily understand that many modifications may be made without departing from the scope of the invention.

1. A method of preparing oxalic acid (H₂C₂O₄), the method at least comprising the steps of:
(a) providing a metal formate (HCO₂M) containing stream, wherein the metal (M) of the metal formate (HCO₂M) is a monovalent metal selected from the group consisting of Li, Na, K, Cs, Rb and a mixture thereof;
(b) heating the metal formate (HCO₂M) containing stream thereby obtaining a metal oxalate (M₂C₂O₄) containing stream;
(c) subjecting the metal oxalate (M₂C₂O₄) containing stream to electrodialysis, thereby obtaining at least oxalic acid (H₂C₂O₄) and a metal hydroxide (MOH).

2. The method according to claim 1, wherein the metal (M) of the metal formate (HCO₂M) is selected from the group consisting of K, Cs, Rb and a mixture thereof.

3. The method according to claim 1, wherein the metal formate (HCO₂M) is potassium formate (HCO₂K).

4. The method according to claim 1, wherein the metal formate (HCO₂M) has been obtained from a metal bicarbonate (MHCO₃).

5. The method according to claim 4, wherein the metal bicarbonate (MHCO₃) has been obtained from CO₂ and a metal hydroxide (MOH).

6. The method according to claim 5, wherein the metal hydroxide (MOH) obtained in step (c) is recycled for use in obtaining the metal bicarbonate (MHCO₃).

7. The method according to claim 1, wherein the metal oxalate (M₂C₂O₄) containing stream subjected in step (c) comprises at most 10.0 wt. % (based on dry matter) carbonate.

8. The method according to claim 1, wherein the oxalic acid (H₂C₂O₄) obtained in step (c) is reacted with hydrogen (H₂) to obtain ethylene glycol (HOCH₂CH₂OH).

9. A method of preparing ethylene glycol (HOCH₂CH₂OH) from CO₂, the method at least comprising the steps of:
(i) reacting CO₂ and a metal hydroxide (MOH) thereby obtaining a metal bicarbonate (MHCO₃), wherein the metal (M) of the metal hydroxide (MOH) is a monovalent metal selected from the group consisting of Li, Na, K, Cs, Rb and a mixture thereof;
(ii) reacting the metal bicarbonate (MHCO₃) obtained in step (i) with hydrogen (H₂) thereby obtaining a metal formate (HCO₂M);
(iii) heating the metal formate thereby obtaining a metal oxalate (M₂C₂O₄);
(iv) subjecting the metal oxalate (M₂C₂O₄) to electrodialysis, thereby obtaining at least oxalic acid (H₂C₂O₄) and a metal hydroxide (MOH);
(v) reacting the oxalic acid (H₂C₂O₄) obtained in step (iv) with hydrogen (H₂) to obtain ethylene glycol (HOCH₂CH₂OH).

10. The method according to claim 9, wherein at least part of the metal hydroxide (MOH) obtained in step (iv) is recycled for use in step (i).
